(19) United States
(12) Patent Application Publication
(10) Pub. No.: US 2024/0358616 A1
(43) Pub. Date: Oct. 31, 2024

(54) Skincare Composition

(71) Applicant: Susan Blyskal, Myrtle Beach, SC (US)

(72) Inventor: Susan Blyskal, Myrtle Beach, SC (US)

(21) Appl. No.: 18/355,430

(22) Filed: Jul. 20, 2023

Related U.S. Application Data
(60) Provisional application No. 63/461,930, filed on Apr. 26, 2023.

(51) Int. Cl. A61K 8/67; A61K 8/02; A61K 8/34; A61K 8/37; A61K 8/46; A61K 8/92; A61Q 19/00

(52) U.S. Cl. CPC A61K 8/678; A61K 8/0216; A61K 8/342; A61K 8/37; A61K 8/463; A61K 8/92; A61Q 19/00

(57) ABSTRACT

A skincare composition that is used to prevent and heal stretch marks and scars and treat dry and flaky skin and other skin ailments is disclosed. The skincare composition is easy-to-use and contains natural and organic components. The composition includes Vitamin E, Coco butter, unrefined Shea Butter, cetyl alcohol, methylparaben, propylene glycol, propylparaben, purified water as a carrier base, sodium lauryl sulfate, stearyl alcohol, aloe vera, and an infused scent. The skincare composition can be applied to any area of the body for moisturizing and hydrating skin and enhancing healing of stretch marks and scars. The composition can be used by individuals of all ages and genders.

### CROSS-REFERENCE TO RELATED APPLICATION

The present application claims priority to, and the benefit of, U.S. Provisional Application No. 63/461,930, which was filed on Apr. 26, 2023 and is incorporated herein by reference in its entirety.

### FIELD OF THE INVENTION

The present invention generally relates to the field of skincare products. More specifically, the present invention relates to a novel skin cream for treating scars, dry and flaky skin, stretch marks, and other skin ailments. The skin cream has a composition combining water-based ingredients and different oils (i.e., Vitamin E oil), butters, and preservatives. The composition can be in the form of a cream, ointment, or lotion and can be easily applied on any part of body by individuals of all ages and genders. Accordingly, the present disclosure makes specific reference thereto. Nonetheless, it is to be appreciated that aspects of the present invention are also equally applicable to other like applications, devices, and methods of manufacture.

### BACKGROUND

By way of background, individuals commonly suffer from irritated, flaky, or cracked skin which can be caused due to different reasons including but not limited to a medical condition such as eczema, dermatitis, and the like. Use of harsh chemicals included in different products such as skincare products, soaps, detergents, and even some cosmetics can irritate the skin and strip it of its natural moisture resulting in flaky and cracked skin. Environmental factors such as cold and windy conditions can cause the skin to lose moisture and become dry and irritated. Similarly, low-humidity climates can lead to dry skin because the air lacks moisture. Without proper treatment, irritated, flaky, or cracked skin can cause significant discomfort and even pain.

Many individuals try different lotions, moisturizers, ointments, and creams to moisturize their skin and alleviate their skin ailments. However, conventional products may contain chemicals that can cause harm and potentially have long-term side effects.

In cases of pregnant women, stretch marks are common on the stomach, arms, legs, thighs, and buttock areas. Stretch marks can occur due to stretching of the skin during pregnancy. Creams and ointments for stretch mark prevention or reduction contain chemical ingredients that can cause potential harm to the pregnant women and developing fetus.

While conventional creams, lotions, moisturizers, and more are commonly used for treating dry and flaky skin, stretch marks, and other ailments, however, they can contain chemicals with potential long-term side effects. Individuals desire a more natural and organic solution for treating dry and flaky skin, stretch marks, and other skin ailments.

Therefore, there exists a long felt need in the art for an improved skin cream, ointment, or lotion for treating dry and flaky skin, stretch marks, and other skin ailments. There is also a long felt need in the art for an improved skin cream that contains organic and natural ingredients. Additionally, there is a long felt need in the art for an improved skin cream or lotion that can be topically applied to any affected area of skin for treating irritated, flaky, stretch marked and cracked skin. Moreover, there is a long felt need in the art for a skin treatment cream that can be used for treating a plurality of skin diseases. Further, there is a long felt need in the art for a skin ointment that provides a pleasant fragrance and soothing relief to uncomfortable areas of skin. Furthermore, there is a long felt need in the art for an improved skin cream that can be applied two times per day for improving skin. Finally, there is a long felt need in the art for an improved skin cream that offers a simple and convenient cream infused with natural ingredients and pleasant scent and can be applied to any area of the body to obtain relief.

The subject matter disclosed and claimed herein, in one embodiment thereof, comprises a composition for treating dry and flaky skin, stretch marks, and other skin ailments in an individual. The composition further comprises Vitamin E, Coco butter, unrefined Shea Butter, cetyl alcohol, methylparaben, propylene glycol, propylparaben, purified water as a carrier base, sodium lauryl sulfate, stearyl alcohol, aloe vera, and an infused scent. The composition can be used by individuals of all ages and genders and can be applied to any part of the skin and enhances prevention and healing of stretch marks and scars while offering soothing relief to uncomfortable areas of skin. Users can apply the cream once or twice per day to the affected area and obtain relief.

In this manner, the novel skincare composition of the present invention accomplishes all of the forgoing objectives and provides users with a relatively safe, easy, cost-effective, and effective solution for preventing stretch marks and treating cracked and flaky skin and other skin ailments. The skin care product of the present invention is also user friendly, inasmuch as it is less expensive, more natural, and organic than other products heretofore known. The skin care product enhances the healing of stretch marks and scars while offering soothing relief to uncomfortable areas of skin and can be applied once or twice per day.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a composition for treating dry and flaky skin, stretch marks, and other skin ailments in an individual. The composition further comprises Vitamin E, Coco butter, unrefined Shea Butter, cetyl alcohol, methylparaben, propylene glycol, propylparaben, purified water as a carrier base, sodium lauryl sulfate, stearyl alcohol, aloe vera, and an infused scent, wherein the composition is in a select one of the following forms: an emulsion, a lotion, a cream, and a tonic. The composition can be used by individuals of all ages and genders and can be applied to any part of the skin.

In a further embodiment of the present invention, a method of treating dry and flaky skin, stretch marks, and other skin ailments of both men and women is disclosed and comprises the steps of contacting an individual's skin where treatment is desired with a skincare composition comprising: 12-20% Vitamin E, 30-35% Coco butter, 20-25% Unrefined Shea butter, 1-2% Cetyl alcohol, 1-3% Methylparaben, 1-2% Propylene glycol, 1-2% Propylparaben, 1-2% Sodium lauryl sulfate, 1-2% Stearyl alcohol, 20-30% Aloe vera liquid, 1-4% scent material, and purified water.

In a further embodiment of the present invention, a composition of use in the treatment of stretch marks and treating cracked and flaky skin comprises, by weight of the composition, 10-15% Vitamin E. 20-25% Coco butter, 25-30% Unrefined Shea butter, 1-3% Cetyl alcohol, 2-3% Methylparaben, 2% Propylene glycol, 2% Propylparaben, 2-3% Sodium lauryl sulfate, 0.5-2.5% Stearyl alcohol, 25-32% Aloe vera liquid, 0.5-2% scent material, and purified water.

The composition of the present invention does not require animal testing and contains the majority of natural and organic components. The composition is readily absorbed in the skin and helps in healing skin naturally.

The advantage of the skincare composition of the present invention is that it provides users with an improved skin cream designed to prevent stretch marks and treat cracked and flaky skin. The ingredients act in harmony with each other to achieve the desired results of healing skin. The cream can be topically applied at room temperature and provides relaxation, cooling, and moisture to the skin surface. Further, the composition is not harmful for pregnant women and a developing fetus. A user can apply a small amount of the composition multiple times every day.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a diagrammatic representation of the components of the skin treatment cream of the present invention used to prevent stretch marks and treat cracked and flaky skin;

FIG. 2 illustrates a user applying the skincare cream product wherein the cream is stored in a small container, as per an exemplary embodiment; and

FIG. 3 illustrates components of a kit in which the skincare composition cream product can be marketed and commercially available.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an improved skin cream, ointment, or lotion for treating dry and flaky skin, stretch marks, and other skin ailments. There is also a long felt need in the art for an improved skin cream that contains organic and natural ingredients. Additionally, there is a long felt need in the art for an improved skin cream or lotion that can be topically applied to any affected area of skin for treating irritated, flaky, stretch marks and cracked skin. Moreover, there is a long felt need in the art for a skin treatment cream that can be used for treating a plurality of skin diseases. Further, there is a long felt need in the art of a skin ointment that provides pleasant fragrance and soothing relief to uncomfortable areas of skin. Furthermore, there is a long felt need in the art for an improved skin cream that can be applied two times per day for improving skin. Finally, there is a long felt need in the art for an improved skin cream that offers a simple and convenient cream infused with natural ingredients and pleasant scent and can be applied to any area of the body to obtain relief.

The present invention, in one exemplary embodiment, is a novel skincare composition for treating dry and flaky skin, preventing stretch marks, and other skin ailments in an individual. The composition further comprises Vitamin E, Coco butter, unrefined Shea Butter, cetyl alcohol, methylparaben, propylene glycol, propylparaben, purified water as a carrier base, sodium lauryl sulfate, stearyl alcohol, aloe vera, and an infused scent, wherein the composition can be topically applied to the skin. The composition can be used by individuals of all ages and genders and can be applied to any part of the skin for healing and rejuvenating the skin.

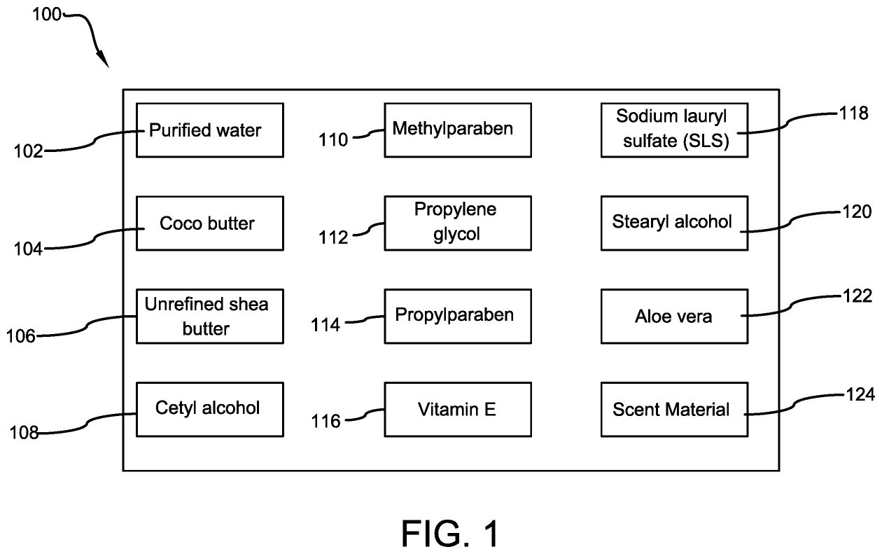

Referring initially to the drawings, FIG. 1 illustrates a diagrammatic representation of the components of the skin treatment cream of the present invention used to prevent stretch marks and treat cracked and flaky skin. The skin treatment cream 100 of the present invention is made up of a majority of natural and organic ingredients which can be derived from natural or commercial sources. All the ingredients provide a unique quality and increase the effectiveness of the skin treatment cream 100 preventing stretch marks and treating cracked, flaky skin and other skin ailments.

Purified water 102 can be in the form of distilled water and forms the solvent for the skin treatment cream 100. Coco butter 104 is used as an emollient and helps to lock in moisture and prevent dehydration of the skin. Further, the coco butter 104 forms a protective barrier on the skin's surface, reducing water loss and keeping the skin hydrated. Coco butter 104 also improves skin elasticity and flexibility, minimizing the formation of stretch marks as the skin stretches. Coco butter 104 also contains antioxidants, such as polyphenols, which protect the skin from oxidative stress and help reduce fine lines and wrinkles. Coco butter 104 used in the cream 100 can be made from cocoa nibs when the cocoa nibs are transformed into chocolate liquor.

Unrefined Shea Butter 106 provides vitamins A, E, and F, as well as essential fatty acids that help nourish and replenish the skin for a healthy and radiant appearance. Unrefined shea butter 106 also has soothing properties to help alleviate skin irritation, inflammation, and itching. Unrefined shea butter 106 has moisturizing and nourishing properties that improve skin elasticity and suppleness to help prevent and minimize the appearance of stretch marks as the skin stretches. In one exemplary embodiment, the Shea butter 106 is derived from the nuts of the shea tree (Vitellaria paradoxa or Vitellaria nilotica) where shea kernels of the nuts are roasted and kneaded to create a thick paste and the paste is boiled or heated to separate the shea butter from the other components.

Cetyl alcohol 108 provides smooth and creamy texture to the skin cream 100 which enables a user to apply the cream 100 easily and smoothly. Cetyl alcohol 108 improves the stability and shelf life of the skin cream product 100 and helps prevent the separation of oil and water-based ingredients (by acting as an emulsifying agent) keeping the cream 100 intact while maintaining effectiveness thereof over time.

Methylparaben 110 is used as a preservative in the skin cream 100. Methylparaben 100 has antimicrobial properties that inhibit the growth of potentially harmful microorganisms in the skin cream 100. This helps in prevention of contamination and spoilage of the skin cream 100 allowing consumers to use the skin cream product 100 for a longer period without compromising the quality thereof.

Propylene glycol 112 functions as a humectant and attracts and retains moisture in the skin cream 100. Propylene glycol 112 also acts as a thickening agent and stabilizer making it easier for the cream 100 to spread and ensuring a smooth application. Propylene glycol 112 also has antimicrobial properties that can help inhibit the growth of bacteria, fungi, and other microorganisms in the skincare cream 100.

Propylparaben 114 is used as a preservative to extend the shelf life and quality of the skin cream product 100. Propylparaben 114 prevents bacterial or fungal infections while applying the cream 100 on skin.

Vitamin oils (i.e., Vitamin E oil) 116 helps to prevent water loss from the skin and maintain hydration of the skin. Vitamin oil also acts as an antioxidant and neutralizes free radicals that are generated by factors such as exposure to UV radiation, pollution, and environmental toxins. Further, Vitamin E oil has anti-inflammatory properties that can help soothe and calm irritated or sensitive skin and helps in reducing the appearance of scars and stretch marks, and prevention of same. In some embodiments, Vitamin E 116 can be obtained from natural sources such as vegetable oil including one of wheat germ oil, sunflower oil, safflower oil, almond oil, and avocado oil. In some embodiments, Vitamin E 116 can be obtained from fruits like mangoes, kiwi, and papaya. In other embodiments, Vitamin E 116 can be derived from petrochemicals or produced through chemical synthesis.

Sodium lauryl sulfate (SLS) 118 is used as a surfactant and helps to remove dirt, excess oil, and impurities from the skin's surface by creating a rich lather. SLS 118 also enhances the penetration of active ingredients (such as Vitamin E, Shea butter, Coco butter and more) into the skin of a user.

Stearyl alcohol 120 functions as an emollient and prevents moisture loss from the skin of a user. Stearyl alcohol 120 facilitates the absorption of other ingredients into the skin and prevents the separation of oil and water-based ingredients.

Aloe vera 122 provides soothing and calming effects on the skin and helps reduce redness, inflammation, and itching, providing relief and promoting skin healing. Aloe vera 122 contains antioxidants, such as vitamins C and E, which help protect the skin from free radical damage caused by environmental factors and provides prevention from damage of same. Aloe vera 122 can be extracted directly from the Aloe vera plant or any commercially available aloe vera extracts.

Scent material 124 such as essential oils derived from various plant sources are infused in the skincare cream 100 for providing a pleasant scent. In some embodiments, floral extracts, obtained from flowers such as rose, jasmine, or lilac, can be used to provide specific floral scents to skin cream 100. To meet requirements of different users, herbal extracts, derived from plants such as mint, eucalyptus, or thyme, offering herbal or medicinal scents can be used in the skincare product 100. Further, in some other embodiments, synthetic fragrance ingredients can also be used to create specific scents or complex fragrance blends to meet requirements of different users. The skincare product 100 can be in the form of emulsions, lotions, creams, and the like.

The skincare composition of the present invention is safe for humans and has been demonstrated to not cause irritation to the skin even when used multiple times in a day and for a long period of time. The composition of the skincare product 100 can be used effectively on uncomfortable areas of skin for treating dry and flaky skin, stretch marks, and other ailments, thereby eliminating the need for multiple skincare products.

Referring to Table 1, there is disclosed an exemplary skincare product 100 composition with a number of different components in varying quantities in order to achieve a desired result. The skincare product 100, in one potential embodiment, is provided as a cream or an ointment. One such exemplary embodiment of the skincare product 100 is provided as shown in Table 1.

TABLE 1

| | Composition in percentage |
|---|---|
| Component | by weight (% w/w) |
| Vitamin oil (i.e., Vitamin E) | 12-20 |
| Coco butter | 30-35 |
| Unrefined Shea Butter | 20-25 |
| Cetyl alcohol | 1-2 |
| Methylparaben | 1-3 |
| Propylene glycol | 1-2 |
| Propylparaben | 1-2 |
| Sodium lauryl sulfate | 1-2 |
| Stearyl alcohol | 1-2 |
| Aloe vera liquid | 20-30 |
| Scent material | 1-4 |
| Purified water | An amount to make the total 100 |

Table 2, there is disclosed alternative healing remedy skin cream 100 with a number of different components in varying quantities in order to achieve a desired result.

TABLE 2

| | Composition in percentage |
|---|---|
| Component | by weight (% w/w) |
| Vitamin oil (i.e., Vitamin E) | 10-15 |
| Coco butter | 20-25 |
| Unrefined Shea Butter | 25-30 |
| Cetyl alcohol | 1-3 |
| Methylparaben | 2-3 |
| Propylene glycol | 1-2 |
| Propylparaben | 1-2 |
| Sodium lauryl sulfate | 2-3 |
| Stearyl alcohol | 0.5-2.5 |
| Aloe vera liquid | 25-32 |
| Scent material | 0.5-2 |
| Purified water | An amount to make the total 100 |

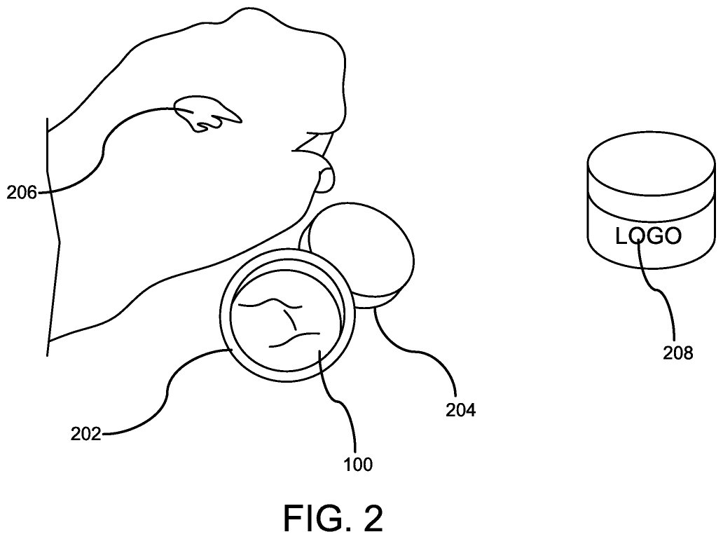

FIG. 2 illustrates a user applying the skincare cream product wherein the cream is stored in a small container, as per an exemplary embodiment. As shown, the cream 100 is stored in container 202 and maintains the consistency and texture. The container 202 can be of different sizers ranging from 8 oz to 40 oz to meet requirements of different users. Further, the container 202 may be of any suitable shape and size, including but not limited to, rectangular, cubic, pyramidal, cylindrical, spherical or any other shape. The containers shown in the figures, and described in the embodiments, are not limited to any particular size or shape. The container 202 can also include a logo, indicia, or trademark 208 along with customizable colors and fonts, embroidery and prints and/or other images on the surface.

A user can take the desired quantity of cream using fingers or a spoon 204 and can apply on the target skin area 206 smoothly and uniformly. In different embodiments, the skincare cream 100 can be applied one time or two times per day. In some embodiments, the cream 100 can also be applied three times per day.

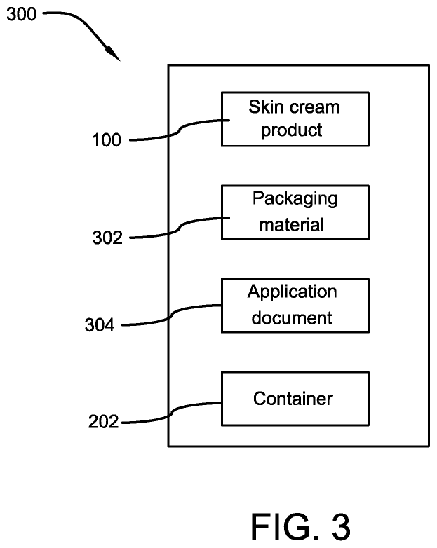

FIG. 3 illustrates components of a kit in which the skincare composition cream product can be marketed and commercially available. As shown, the skincare composition cream product kit 300 includes a packaging material 302 in the form of a box in which the container 202 is secured. The packaging material 302 can have a logo, trademark, and ingredients on the surface. A direction for application document 304 can also be included in the kit 300 to help a user in applying the cream composition 100. The container 202 contains the cream composition 100 in a sealed manner.

The composition of the present invention is useful as a pharmaceutical or an ayurvedic product and functions as a skincare product for individuals of all ages, and for different skin ailments.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “skincare composition cream product”, “cream composition”, “skin treatment cream”, “skin cream”, and “cream” are interchangeable and refer to the skincare composition cream product 100 of the present invention.

Notwithstanding the forgoing, the skincare composition cream product 100 of the present invention can contain any additional ingredient known in the art without affecting the overall concept of the invention, provided that it accomplishes the above-stated objectives. One of ordinary skill in the art will appreciate that the ingredients and composition of the skincare composition cream product 100 as shown in the FIGS. are for illustrative purposes only, and that many other compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A skin care ointment composition comprising an ointment composition selected from a group consisting of one or more of a Vitamin oil, a Coco butter, an unrefined shea butter, a cetyl alcohol, a methylparaben, a propylene glycol, a propylparaben, a sodium lauryl sulfate, a stearyl alcohol, an aloe vera liquid, a scent material, and a purified water.

2. The skin care ointment composition of claim 1, wherein said skin care ointment is in a form selected from a lotion, an emulsion, and a cream.

3. The skin care ointment composition of claim 2, wherein said scent material is selected from a group consisting of one or more of an essential oil, a floral extract, and an herbal extract.

4. The skin care ointment composition of claim 3, wherein said floral extract is selected from a group consisting of one or more of a rose, a jasmine, and a lilac.

5. The skin care ointment composition of claim 3, wherein said herbal extract is selected from a group consisting of one or more of a mint, a eucalyptus, and a thyme.

6. The skin care ointment composition of claim 3, wherein said Vitamin oil is Vitamin E oil from 12 to 20 percent by weight of said ointment composition.

7. The skin care ointment composition of claim 3, wherein said Coco butter is from 30 to 35 percent by weight of said ointment composition.

8. The skin care ointment composition of claim 3, wherein said unrefined shea butter is from 20 to 25 percent by weight of said ointment composition.

9. The skin care ointment composition of claim 3, wherein said cetyl alcohol is from 1 to 2 percent by weight of said ointment composition.

10. The skin care ointment composition of claim 3, wherein said methylparaben is from 1 to 3 percent by weight of said ointment composition.

11. The skin care ointment composition of claim 3, wherein said propylene glycol is from 1 to 2 percent by weight of said ointment composition.

12. The skin care ointment composition of claim 3, wherein said propylparaben is from 1 to 2 percent by weight of said ointment composition.

13. The skin care ointment composition of claim 3, wherein said sodium lauryl sulfate is from 1 to 2 percent by weight of said ointment composition.

14. The skin care ointment composition of claim 3, wherein said stearyl alcohol is from 1 to 2 percent by weight of said ointment composition.

15. The skin care ointment composition of claim 3, wherein said aloe vera liquid is from 20 to 30 percent by weight of said ointment composition.

16. The skin care ointment composition of claim 3, wherein said scent material is from 1 to 4 percent by weight of said ointment composition.

17. The skin care ointment composition of claim 3 wherein said purified water is in percent by weight to form a remainder by weight and said ointment composition of 100 percent.

18. A skin care ointment composition comprising an ointment composition selected from a group consisting of one or more of a Vitamin E oil, a Coco butter, an unrefined shea butter, a cetyl alcohol, a methylparaben, a propylene glycol, a propylparaben, a sodium lauryl sulfate, a stearyl alcohol, an aloe vera liquid, a scent material, and a purified water;
wherein said Vitamin E oil is from 10 to 15 percent by weight of said ointment composition;
wherein said Coco butter is from 20 to 25 percent by weight of said ointment composition;
wherein said unrefined shea butter is from 25 to 30 percent by weight of said ointment composition;
wherein said cetyl alcohol is from 1 to 3 percent by weight of said ointment composition;
wherein said methylparaben is from 2 to 3 percent by weight of said ointment composition;
wherein said propylene glycol is from 1 to 2 percent by weight of said ointment composition;
wherein said propylparaben is from 1 to 2 percent by weight of said ointment composition;
wherein said sodium lauryl sulfate is from 2 to 3 percent by weight of said ointment composition;
wherein said stearyl alcohol is from 0.5 to 2.5 percent by weight of said ointment composition;
wherein said aloe vera liquid is from 25 to 32 percent by weight of said ointment composition;
wherein said scent material is from 0.5 to 2 percent by weight of said ointment composition; and
further wherein said purified water is in percent by weight to form a remainder by weight and said ointment composition of 100 percent.

19. The skin care ointment composition of claim 18, wherein said scent material is selected from a group consisting of one or more of an essential oil, a floral extract, and an herbal extract, wherein said floral extract is selected from a group consisting of one or more of a rose, a jasmine, and a lilac, and further wherein said herbal extract is selected from a group consisting of one or more of a mint, a eucalyptus, and a thyme.

20. A skin care ointment composition comprising:
an ointment composition selected from a group consisting of one or more of a Vitamin oil, a Coco butter, an unrefined shea butter, a cetyl alcohol, a methylparaben, a propylene glycol, a propylparaben, a sodium lauryl sulfate, a stearyl alcohol, an aloe vera liquid, a scent material, and a purified water;
wherein said Vitamin oil is from 12 to 20 percent by weight of said ointment composition;
wherein said Coco butter is from 30 to 35 percent by weight of said ointment composition;
wherein said unrefined shea butter is from 20 to 25 percent by weight of said ointment composition;
wherein said cetyl alcohol is from 1 to 2 percent by weight of said ointment composition;
wherein said methylparaben is from 1 to 3 percent by weight of said ointment composition;
wherein said propylene glycol is from 1 to 2 percent by weight of said ointment composition;
wherein said propylparaben is from 1 to 2 percent by weight of said ointment composition;
wherein said sodium lauryl sulfate is from 1 to 2 percent by weight of said ointment composition;
wherein said stearyl alcohol is from 1 to 2 percent by weight of said ointment composition;
wherein said aloe vera liquid is from 20 to 30 percent by weight of said ointment composition;
wherein said scent material is from 1 to 4 percent by weight of said ointment composition; and
further wherein said purified water is in percent by weight to form a remainder by weight and said ointment composition of 100 percent.
